(19) United States
(12) Patent Application Publication
(10) Pub. No.: US 2011/0106201 A1
(43) Pub. Date: May 5, 2011

(54) IMPLANTABLE HEART FAILURE MONITOR

(76) Inventor: Sourav Bhunia, Shoreview, MN (US)

(21) Appl. No.: 12/609,479

(22) Filed: Oct. 30, 2009

(51) Int. Cl. A61N 1/365; A61B 5/1459

(52) U.S. Cl. 607/18; 600/309; 600/339; 600/333; 600/301

(57) ABSTRACT

An implantable medical device and associated method monitor a heart failure patient by sensing a signal responsive to oxygen availability in an extravascular volume of skeletal muscle tissue. The signal is used to compute a tissue oxygenation measurement. A change in the tissue oxygenation measurement is detected, and a time interval corresponding to the detected change in muscle tissue oxygenation is computed. The time interval is used for detecting if a heart failure condition is worsening or improving.

### TECHNICAL FIELD

The disclosure relates generally to implantable medical devices and, in particular, to an implantable medical device and associated method for monitoring a heart failure patient.

### BACKGROUND

Patients suffering from heart failure can experience severe symptoms leading to hospitalization as their heart failure worsens. It is desirable to prevent hospitalization and worsening heart failure symptoms by managing medications and other heart failure therapies, such as cardiac resynchronization therapy (CRT). However, clinicians are challenged in detecting a worsening state of heart failure patient before the patient becomes overtly symptomatic and hospitalization is required. Implantable hemodynamic monitors (IHMs) are available which monitor hemodynamic signals of a patient for detecting or predicting a worsening hemodynamic condition. However, a need remains for a medical device and method for ambulatory monitoring of heart failure patients which allows early detection of a worsening heart failure condition.

### BRIEF DESCRIPTION OF THE DRAWINGS

FIG. 1 is a schematic diagram of an implantable medical device (IMD) for monitoring a heart failure patient.

FIG. 2 is a functional block diagram of an IMD associated with an optical sensor for monitoring tissue oxygenation.

FIG. 3 is an illustrative plot of a tissue oxygenation measurement over time in response to aerobic exercise.

FIG. 4 is a flow chart of a method for monitoring a heart failure patient.

FIG. 5 is a plot of a tissue oxygenation measurement illustrating an alternative method for measuring a recovery curve slope for use in computing an exercise tolerance metric.

FIG. 6 is a hypothetical plot of an exercise tolerance metric used for monitoring a patient's heart failure condition.

FIG. 7 is a hypothetical plot of recovery time interval data acquired over three different time periods for spontaneously detected activity levels.

FIG. 8 is a flow chart of a method for managing a heart failure therapy.

### DETAILED DESCRIPTION

In the following description, references are made to illustrative embodiments. It is understood that other embodiments may be utilized without departing from the scope of the disclosure. In some instances, for purposes of clarity, identical reference numbers may be used in the drawings to identify similar elements. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

In various embodiments described herein, a physiological sensor is used to monitor oxygenation of skeletal muscle tissue in response to exercise. The sensor may be embodied as an optical sensor emitting light and detecting light scattered and reflected by an extravascular tissue measurement volume back to the sensor. The extravascular measurement volume for an optical sensor is the volume of tissue (including blood) in the sensing path of the sensor. The term “tissue oxygenation” as used herein refers to the availability of oxygen to a localized tissue volume and thus refers generally to the availability of oxygenated hemoglobin. Generally speaking, the availability of oxygen to skeletal muscle tissue is being monitored. As such, the optical sensor is positioned so that the extravascular measurement volume of the sensor extends through a relatively uniform volume of skeletal muscle tissue containing microvessels present in the muscle tissue.

The sensor itself may be positioned intravascularly or extravascularly as long as the measurement volume includes an extravascular volume of skeletal muscle tissue and is not limited to a blood volume when positioned intravascularly. For example, an optical sensor may be positioned along a blood vessel wall “looking out” toward a volume of skeletal muscle tissue. By using an extravascular sensor, complications associated with positioning a sensor within the blood stream can be avoided.

The status of a heart failure patient may be monitored by assessing the tissue oxygenation during exercise and/or during a post-exercise recovery period. Generally speaking, as heart failure worsens, tissue oxygenation worsens. As heart failure improves, for example in response to a heart failure therapy, tissue oxygenation also improves. In particular, a heart failure patient's exercise tolerance becomes impaired as cardiac reserve is diminished. Diminished cardiac reserve is a reduced ability of the heart to respond to an increased metabolic demand by increasing cardiac output. As a result, the tissue oxygen availability does not increase sufficiently to meet an increased metabolic demand due to exercise. A method and apparatus for monitoring the heart failure status of a patient by monitoring tissue oxygenation is described here. The methods and apparatus described herein may also be useful in monitoring diseases or conditions other than heart failure that affect skeletal muscle oxygenation and exercise tolerance.

Tissue oxygenation during exercise may be lower in a heart failure patient or decrease more rapidly during exercise as compared to a patient having normal cardiac function performing the same level of exercise. Tissue oxygenation during a post-exercise recovery period may recover (increase) at a slower rate in a heart failure patient as compared to a healthy patient. Accordingly, a trend in the rate of exercise-induced decrease in tissue oxygenation and/or a trend in the rate of post-exercise increase in tissue oxygenation may be used to detect a worsening or improving heart failure condition. A faster exercise induced decrease and/or a slower post-exercise recovery for an equivalent exercise level would indicate a worsening condition and would be associated with worsening tolerance for exercise in the heart failure patient, i.e., a worsening heart failure condition. Likewise a slower decrease during exercise and/or faster recovery post-exercise would indicate an improving heart failure condition.

An optical sensor or other sensor used to monitor tissue oxygenation in an extravascular measurement tissue volume may be a calibrated sensor providing an absolute oxygen saturation measurement or an uncalibrated sensor providing an index of oxygen saturation as a measure of tissue oxygenation. Tissue oxygenation monitoring as implemented in a device and associated method as described herein may involve, without limitation, measuring absolute oxygen saturation, total hemoglobin volume fraction, an oxygen saturation index, direct measurement of tissue oxygen partial pressure, or any combination thereof.

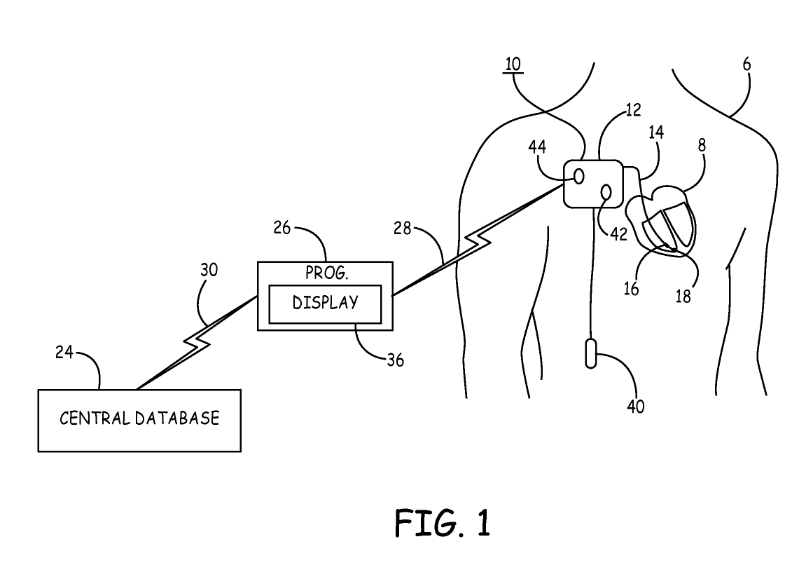

FIG. 1 is a schematic diagram of an implantable medical device (IMD) 10 optionally coupled to a lead 14 positioned within a heart 8 in a patient's body 6. IMD 10 may correspond to a variety of implantable medical devices including a cardiac pacemaker, implantable cardioverter defibrillator, implantable hemodynamic monitor, a drug pump, a neurostimulator or the like. IMD 10 may or may not be provided with therapy delivery capabilities. Accordingly, IMD 10 may be coupled to additional leads and/or catheters operatively positioned relative to the patient's heart 8 or other body tissues for deploying stimulating/sensing electrodes, other physiological sensors, and/or drug delivery ports. While lead 14 is shown terminated within the right ventricle of the patient's heart, it is recognized that lead 14 may be configured as a transvenous that extends into other heart chambers or blood vessels or a subcutaneous lead extending to other body locations for positioning electrodes and/or physiological sensors in a desired location.

In one embodiment, IMD 10 corresponds to an implantable heart failure monitor capable of at least sensing a signal corresponding to patient activity and a signal corresponding to tissue oxygenation. IMD 10 stores the sensed signals and derives a trend in the patient's heart failure condition from the sensed signals.

Housing 12 encloses circuitry (not shown in FIG. 1) included in IMD 10 for controlling and performing device functions and processing sensed signals. An activity sensor 44 may be enclosed in housing 12 for use in monitoring patient activity. An optical sensor 42 may be incorporated along housing 12 for sensing tissue oxygenation of an adjacent tissue volume. Alternatively or additionally, one or more optical sensor(s) 40 may be implanted remotely from IMD 10 for monitoring tissue oxygenation at a different implant location than the IMD 10 implant location. Sensor 40 may be carried by a lead or may be implemented as a wireless sensor in telemetric communication with IMD 10 and/or external device 26. For example, it may be desirable to implant a tissue oxygenation sensor 40 in a limb along a major muscle group utilized during patient exercise, such as along a calf, thigh, or upper arm. Alternatively sensor 40 or 42 may be implanted in a position to measure tissue oxygenation along a core body muscle, for example along the thorax, abdomen or back.

In the illustrative embodiments described herein, an optical sensor is used for monitoring tissue oxygenation for use in determining trends in activity-related tissue deoxygenation and oxygen recovering rates. However other sensors generating a signal correlated to tissue oxygenation may be substituted for the optical sensor described herein.

IMD 10 may additionally sense and record intracardiac electrogram (EGM) or electrocardiogram (ECG) signals, intracardiac pressure signals, transthoracic impedance signals, heart wall motion, heart sounds, or other signals useful in monitoring heart failure. For example, lead 14 may include one or more electrodes 18 for sensing cardiac EGM signals and other physiological sensors, such as a pressure sensor 16, for monitoring intracardiac pressure.

IMD 10 is capable of bidirectional communication with an external device 26 via bidirectional telemetry link 28. Device 26 may be embodied as a programmer, typically located in a hospital or clinic, used to program the operating mode and various operational variables of IMD 10 and interrogate IMD 10 to retrieve data acquired and stored by IMD 10. Device 26 may alternatively be embodied as a home monitor used for remote patient monitoring for retrieving data from the IMD 10 and may be used to program IMD 10 but possibly with limited programming functionality as compared to a hospital or clinic programmer. Data stored and retrieved from IMD 10 may include data related to IMD function determined through automated self-diagnostic tests as well as physiological data acquired by IMD 10 including patient activity and tissue oxygenation data used to assess the patient's response to exercise as a method for monitoring heart failure status.

External device 26 is further shown in communication with a central database 24 via communication link 30, which may be a wireless or hardwired link. Programming data and interrogation data may be transmitted via link 30. Central database 24 may be a centralized computer, Internet-based or other networked database used by a clinician for remote monitoring and management of patient 6. An example of a remote patient management system in which tissue oxygenation monitoring may be incorporated for monitoring heart failure patients is generally described in commonly-assigned U.S. Pat. No. 6,599,250 (Webb, et al.), hereby incorporated herein by reference in its entirety. It is recognized that other external devices, such as other physiological monitoring devices or other types of programming devices, may be used in conjunction with IMD 10 and incorporate portions of the methods described herein.

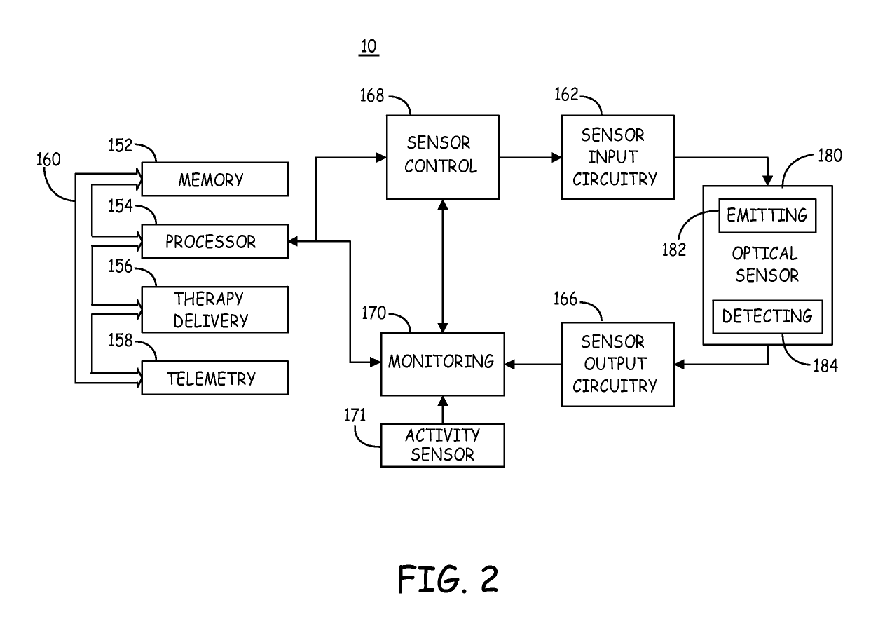

FIG. 2 is a functional block diagram of an IMD 10 associated with an optical sensor 180 for monitoring tissue oxygenation. IMD 10 shown includes (or is coupled to) an optical sensor 180, which may be incorporated in or on a hermitically sealed housing of IMD 10, carried by a lead extending from IMD 10, or embodied as a wireless sensor in telemetric communication with IMD 10. IMD 10 further includes sensor input circuitry 162 and sensor output circuitry 166.

Optical sensor 180 generally includes a light emitting portion 182 including a light source for emitting light through blood-perfused, skeletal muscle tissue of the patient and a light detecting portion 184 including a light detector, also referred to herein as a “photodetector”, for generating a signal representative of an intensity of light scattered by the muscle tissue to the light detector.

Sensor input circuitry 162 is coupled to light emitting portion 182 of optical sensor 180 and provides the drive signals applied to the light source(s) included in light emitting portion 182 to cause controlled light emission, e.g. controlled intensity, time duration and frequency. Light emitting portion 182 includes one or more light sources for emitting light at two or more different wavelengths. Light sources may include separate light sources that emit light at discrete wavelengths or a single broad spectrum or white light source may be used.

Deoxygenation and reoxygenation of blood may be monitored at various sites with a sensor implanted in a human. The implant site may influence the oxygenation parameters that may be monitored. Loss of blood oxygen takes place at capillaries and the resulting deoxygenated blood is convected downstream to the veins leading to a venous oxygen level lower than the arterial level. At the onset of exercise, capillaries of the skeletal muscles participating in the exercise undergo additional deoxygenation. The resulting deoxygenated blood mixes with less deoxygenated blood returned to the venous vasculature from the rest of the body. Therefore deoxygenation of the mixed venous blood in the vena cava and right atrium and ventricle may be delayed due to the convection time and may be less pronounced due to mixing. Measurement of skeletal muscle oxygenation may provide a more rapid and more sensitive means of monitoring deoxygenation and reoxygenation of blood associated with physical activity and exercise compared to intravenous and intracardiac measurements of blood oxygen saturation and or partial pressure of oxygen.

A trend in tissue oxygenation can be measured with an implanted extravascular optical sensor with two or more wavelengths of light. The measurement of light intensity for at least two different wavelengths, typically red and infrared, allows an index of oxygen saturation to be computed. Two- and three-wavelength sensors that allow an oxygen saturation index to be computed are useful in measuring a relative change in oxygen availability rather than absolute levels of oxygen saturation. The two- and three-wavelength systems tend to be subject to baseline wander. As such, measurements obtained over relatively short periods of time, such as one minute or less, allow relative changes to be measured with error due to baseline shifts minimized.

The measurement of scattered light for at least four different wavelengths, typically within the red and infrared spectrum, may be used to compute a calibrated oxygen saturation measurement. Measurements at three to eight wavelengths in the red and the infrared spectral regions may also be used to compute trends of the concentrations of the various hemoglobin species and thereby the tissue oxygenation level. For descriptions of sensors and related methods for monitoring an oxygen saturation index or absolute oxygen saturation measurement, reference is made to commonly-assigned U.S. Pat. Publication No. 2007/0255148 (Bhunia), U.S. Pat. Publication No. 2008/0208269 (Cinbis, et al), and U.S. Pat. Application No. XX/XXX,XXX, (Attorney Docket No. P0034665.00), all of which are hereby incorporated herein by reference in their entirety.

Sensor input circuitry 162 is controlled by sensor control module 168 which coordinates the beginning time, duration, and frequency of drive signals produced by sensor input circuitry 162. Emitted light may be controlled according to different light modulation schemes, such as a time or frequency multiplexed schemes to allow the intensity of separate wavelengths received by detecting portion 184 to be measured.

Sensor output circuitry 166 receives the photodetector signal from light detecting portion 184 and demodulates and digitizes the signal to provide a digital signal to monitoring module 170. Sensor output circuitry 166 may include an analog-to-digital converter and memory for digitizing an analog output signal from detecting portion 184, providing the digitized signal to monitoring module 170, storing measurement results for future retrieval as well as storing calibration coefficients or other coefficients used in computing calibrated or indexed values of a tissue oxygenation measurement.

Monitoring module 170, in response to patient activity sensed by activity sensor 171, uses the optical signal to compute a tissue oxygenation measurement using the intensities of the light measured by detecting portion 184. The tissue oxygenation measurements are then used by processor 154 to compute a time interval corresponding to deoxygenation during exercise or during post-exercise oxygen recovery. The time interval may be used as a metric of the patient's exercise tolerance. Trends in the time interval or a corresponding slope of the oxygenation measurement over an identified time interval are determined to detect a worsening or improving heart failure condition.

Therapy delivery module 156 may be included having electrical pulse generation capabilities for delivering cardiac pacing pulses and cardioversion/defibrillation shocks. Therapy delivery module 156 may additionally include a fluid delivery pump for delivering a pharmaceutical or biological fluid to the patient and/or provide nerve stimulation therapy.

Data acquired by processor 154 relating to tissue oxygenation and activity measurements may be stored in memory 152 and/or transferred to a medical device programmer, home monitor, computer, or other external or bedside medical device via wireless telemetry module 158 for review by a clinician. Processor 154 transmits data to and from memory 152, therapy delivery module 156, and telemetry module 158 via data/address bus 160.

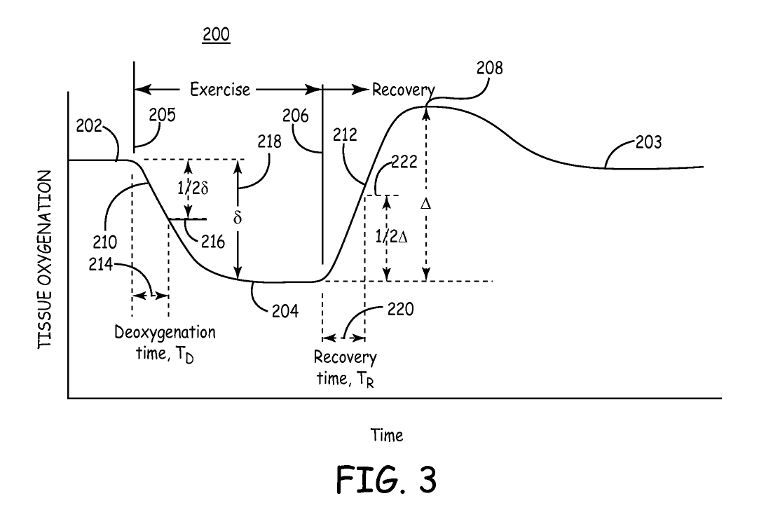

FIG. 3 is an illustrative plot of a tissue oxygenation measurement over time in response to physical exercise. The tissue oxygenation measurement is plotted on the y-axis over time plotted along the x-axis. The tissue oxygenation measurement is at an initial resting, baseline value 202. Upon the onset of exercise at 205, tissue oxygenation begins to decrease. The tissue oxygenation measurement decreases during exercise and may reach a plateau 204, lower then the initial resting level 202, during a sustained level of aerobic exercise. When the patient stops exercising at 206, the tissue oxygenation recovers. Tissue oxygenation increases back to the resting level 203, sometimes overshooting a final resting baseline 203 and reaching a maximum peak tissue oxygenation 208. Depending on the type of sensor used and other physiological factors, the post-exercise resting baseline 203 may or may not be equal to the pre-exercise resting baseline 202.

In a patient suffering from worsening heart failure, the rate of deoxygenation of the tissue during exercise, as represented by deoxygenation curve 210, increases as the patient's cardiac reserve becomes more and impaired. The rate of recovery of the tissue oxygenation measurement after exercise, as shown by the recovery curve 212, becomes slower. As such, a slope of the deoxygenation curve 210 and/or a slope of the recovery curve 212 may be used as a metric of the patient's exercise tolerance. Trends in these slopes, individually or combined in an exercise tolerance metric, may be evaluated to detect a worsening or improving heart failure condition.

In one embodiment, the difference 218 between the resting plateau 202 and the non-resting plateau 204 (during sustained exercise) is determined. The time 214 to reach the half-way point 216 (or other percentage) between the resting plateau 202 and the non-resting plateau 204 is determined as a deoxygenation time. Alternatively, the deoxygenation time may be determined as the time interval between reaching two pre-determined oxygenation levels between the resting plateau 202 and the non-resting plateau 204, for example the 25^{th} and the 75^{th} percentage levels of the range between the two plateaus 202 and 204.

Alternatively, the deoxygenation time may be determined as the time interval between reaching two pre-determined oxygenation levels between the oxygenation level when the non-resting activity level is first detected, and the subsequent non-resting oxygenation plateau 204. The predetermined levels may be the oxygenation level at the onset of exercise at 205 and a predetermined percentage, for example 50%, of the range between the oxygenation level at onset and the subsequent non-resting oxygenation plateau 204.

Depending on the sensor being used to acquire oxygenation measurements, baseline drift may reduce the validity of historical resting baseline measurements acquired at optional block 305 for determining a deoxygenation time. As such, an onset oxygenation measurement taken at the onset of exercise 205 may provide a reference level for use in determining a deoxygenation time. It is recognized that a reference level measurement may include sampled measurements acquired over a short time interval just prior to and/or subsequent to the exercise onset. The deoxygenation time for a known non-resting activity level can be determined as a metric of exercise tolerance when a deoxygenation plateau is reached.

Additionally or alternatively, a recovery time 220 may be determined as a metric of exercise tolerance. The recovery time 220 is determined as the time to reach the half-way point 222 (or other percentage) between the non-resting plateau 204 and either a post-exercise tissue oxygenation peak 208 or the post-exercise resting plateau 203. Alternatively, the recovery time may be determined as the time interval between reaching two pre-determined oxygenation levels between the non-resting plateau 204 and either a post-exercise tissue oxygenation peak 208 or the post-exercise resting plateau 203, for example the 25^{th} and the 75^{th} percentage levels of the range between the two reference levels. Alternatively, the recovery time interval may be determined as the time for the tissue oxygenation measurement to increase from its lowest level during the period of activity to a predetermined percentage of the range between that level and the subsequent peak 208 or the post-exercise resting plateau, 203.

An exercise tolerance metric may be computed as either the tissue deoxygenation time 214 or the tissue recovery time 220 or a mathematical combination of both. For an episode of sustained activity above the non-resting level, there may be a measurable tissue deoxygenation time 214 depending on whether a reference deoxygenation plateau is reached and how the deoxygenation time is being computed. There will be a measurable tissue recovery time 220 as long as the level of oxygenation decreased below the resting level 202 without necessarily reaching a non-resting plateau 204.

Depending on the sensor used, the initial resting level 202 and post-exercise resting baseline 203 and even the absolute levels of the plateau 204 and peak 208 may be subject to baseline drift such that these levels themselves do not provide reliable absolute measures. However relative changes in indexed measurement that occur over relatively short periods of time, for example less than approximately one minute, may be measured accurately using a two- or three-wavelength sensor. As such, a differential measurement along the deoxygenation curve 210 and/or along the recovery curve 212 may be used as a measurement for computing an exercise tolerance metric.

Using a slope of the deoxygenation curve 210 or recovery curve 212 measured in an extravascular measurement volume allows measurements to be made without requiring knowledge of a pre-exercise or post-exercise oxygen availability measurement. Furthermore, knowledge of the time that the exercise has stopped is not necessary since a recovery curve slope can be measured based on detecting an increasing trend in the indexed measurement after detecting either the onset of exercise 205, after detecting a decreasing trend in the indexed measurement, or after detecting a minimum value of the indexed measurement. An exercise plateau 204 may never occur depending on the type of activity and condition of the patient. A differential measurement based on a minimum measurement and one or more measurements along the recovery curve 212 may be used for computing an exercise tolerance metric. Activity or exercise detection is not required for setting intervals over which oxygen availability measurements are made even though the measurements may ultimately be analyzed in combination with measured activity levels of the patient.

Since the extravascular measurement is performed in a skeletal muscle tissue volume the measurement has a quicker response time to exercise than changes in venous blood and may be more closely correlated to the actual oxygen availability to the exercising tissue since a venous blood measurement may represent other systemic changes as well.

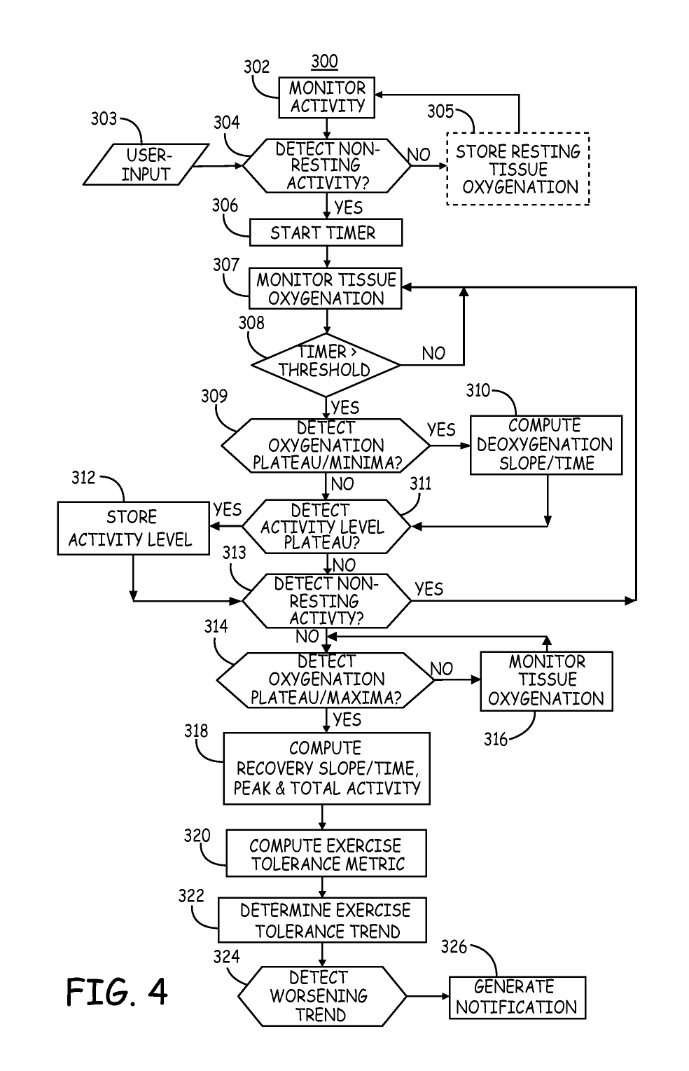

FIG. 4 is a flow chart of a method 300 for monitoring a heart failure patient. Flow chart 300 is intended to illustrate the functional operation of the device, and should not be construed as reflective of a specific form of software or hardware necessary to practice the methods described. It is believed that the particular form of software will be determined primarily by the particular system architecture employed in the device. Providing software to accomplish the described functionality in the context of any modern IMD, given the disclosure herein, is within the abilities of one skilled in the art.

Methods described in conjunction with flow charts presented herein may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 302, patient activity is monitored using an implantable activity sensor. Patient activity may be monitored using a one-, two- or three-dimensional accelerometer. Two activity parameters, activity level and total activity, may be monitored by integrating the acceleration signal when it is above a threshold, over either a fixed or the entire duration of activity, respectively. Depending on the nature of the activity, the activity level may vary over the duration of activity but there will be one value of the total activity for an episode of activity. Depending on the type of activity the activity level may reach a peak and/or a plateau during a period of activity.

At block 304, a non-resting level of activity is detected. Non-resting activity may be detected based on a threshold activity level applied to the activity sensor signal. The non-resting activity level may correspond to any specified threshold level above a patient's resting baseline. For example, a non-resting activity level detected at block 304 may correspond to some level of exertion associated with exercise or activity more strenuous than activities of daily living (i.e., getting out of bed, getting dressed, moving about the house, etc.).

A threshold set for detecting non-resting activity at block 304 may be programmed by a user or may be computed and updated automatically by the IMD based on trends in patient activity. For example, whenever a patient reaches an upper percentile, for example a seventieth or eightieth percentile, of all activity levels detected for the patient, a non-resting activity level is detected for enabling tissue oxygenation monitoring.

In other embodiments, any activity level detected above a resting baseline signal could be detected at block 304 and may be used to initiate a timer at block 306 and tissue oxygenation monitoring at block 307. It is recognized that a specified level of non-resting activity may be required to be sustained for some minimal interval of time before using tissue oxygenation monitoring at block 307 for determining an exercise tolerance metric.

Additionally or alternatively, non-resting activity is detected at block 304 in response to user input as indicated at block 303. A patient or clinician using an external programmer, home monitor, or hand held device may provide a signal to the IMD that the patient is starting to exercise. For example, a patient may be instructed to perform a prescribed level of exercise on a regular basis. The patient may use an external device to indicate to the IMD that he/she is about to begin the prescribed level of exercise. This allows metrics of exercise tolerance to be computed based on a repeatable, prescribed exercise level. Exercise tolerance metrics acquired for the prescribed exercise level can be plotted over time to determine an overall trend in the exercise tolerance metric as an indicator of heart failure condition.

If a resting level of activity is detected (a negative result at decision block 304), i.e., the activity sensor signal is at a baseline resting level or is below a specified non-resting activity threshold, a baseline tissue oxygenation measurement may be determined and stored at block 305. The stored resting tissue oxygenation measurement may be used by a processor performing an algorithm associated with method 300 for determining a metric of exercise tolerance. A resting baseline or plateau level of the tissue oxygenation measurement may be updated on a periodic or continuous basis whenever a resting activity level is detected and may be averaged over a time interval of sustained rest. However when measuring a relative change in a tissue oxygenation measurement during exercise or during recovery, the baseline tissue oxygenation measurement is not needed.

In response to detecting a non-resting activity level at block 304, tissue oxygenation monitoring is performed at block 307. Prior to detecting a non-resting activity, tissue oxygenation measurements may be obtained on a periodic basis to update the resting tissue oxygenation measurement but continuous tissue oxygenation monitoring may not be needed. When a non-resting level of activity is detected, tissue oxygenation measurements are sampled with increased frequency compared to periods of rest, or continuously, in order to obtain a slope or time interval associated with a change in tissue oxygenation during exercise or during post-exercise recovery.

In response to a sustained level of physical activity, the tissue oxygenation measurement may decline from the resting baseline level to a lower, exercising level or plateau as previously shown in FIG. 3. As such, the tissue oxygenation measurement is monitored at block 307 until a non-resting plateau (a plateau lower than the resting baseline level) is reached, as determined at block 309, after a minimum activity episode duration has passed as determined at block 308. Alternatively, a minimum oxygenation measurement may be detected as a minimum peak of a decreasing deoxygenation curve followed by an increasing reoxygenation curve. Thus, even when a plateau is never reached, a minimum peak of the curve may be used in computing an oxygenation measurement.

If a non-resting plateau level (or minimum peak) of the tissue oxygenation measurement is detected at block 309, the deoxygenation plateau level (or minimum peak) is recorded and a deoxygenation slope or associated time interval may be computed at block 310. The deoxygenation time interval is the time for the tissue oxygenation measurement to fall some predetermined amount during the sustained non-resting activity. The decrease in the tissue oxygenation measurement may be defined relative to the resting baseline tissue oxygenation and/or the non-resting plateau or minimum peak value. For example, the time interval for the tissue oxygenation to fall from the resting baseline to some percentage below the resting baseline may be determined as a deoxygenation time interval. Alternatively, the time interval for the tissue oxygenation to fall from the resting baseline to a half-way point between the resting baseline and a non-resting plateau or other methods as described in conjunction with FIG. 3 may be used to determine the deoxygenation time interval at block 310.

If an activity level plateau has also been reached, as determined at block 311, the activity level is recorded at block 312. If a non-resting plateau level of the tissue oxygenation measurement is not detected at block 309, and the activity level has reached a plateau (block 311), the activity level is recorded at block 312.

In some embodiments, a deoxygenation time interval or slope of the tissue oxygenation measurement curve determined during exercise may be used directly for computing a metric of exercise tolerance without further monitoring of tissue oxygenation. In other embodiments, the tissue oxygenation monitoring may continue to acquire tissue oxygenation measurements during a recovery period after the patient discontinues the sustained exercise or activity.

As long as non-resting activity is still being detected at block 313, the tissue oxygenation measurement continues to be monitored by returning to block 307. If a non-resting activity level is no longer being detected at block 313, i.e. the patient has returned to a resting state, tissue oxygenation monitoring continues at block 316 until a post-exercise resting tissue oxygenation plateau is reached as determined at block 314. Criteria for detecting a non-resting plateau may vary between embodiments but will generally require the tissue oxygenation measurement to remain equal to, or within a predefined range, of a previous measurement for some minimum number of subsequent measurements, which may be consecutive measurements, or some minimal interval of time. Once a post-exercise tissue oxygenation plateau is reached, the oxygenation recovery time or slope is computed at block 318.

A recovery time interval may be determined as the time for the tissue oxygenation measurement to increase by a predetermined amount. The time interval may be measured as the time for the oxygenation measurement to increase from some predetermined level or percentage, defined relative to the non-resting minimum peak or plateau and/or relative to the post-exercise resting baseline, to the detected recovery peak or a restored resting baseline level. In one embodiment, the recovery time interval is determined as the time for the tissue oxygenation measurement to increase from the detected non-resting plateau (or minimum peak) to fifty percent of a subsequent tissue oxygenation peak or resting plateau. This and other methods for computing a recovery time are described in conjunction with FIG. 3.

The recovery time interval (or recovery curve slope) may be used alone for determining a tissue oxygenation metric without determining a deoxygenation time interval. The recovery time interval may be determined without a known pre-exercise resting baseline. When the pre-exercise resting baseline is not needed for computing an exercise metric, tissue oxygenation monitoring may be performed only after a non-resting activity level is detected which would conserve IMD processing power.

If a non-resting activity level is again detected while executing blocks 314 or 316, method 300 may abandon computation of an exercise tolerance metric and return to block 302 to continue monitoring the activity. The patient may have begun exercising or performing another task or activity resulting in a non-resting activity again being detected prior to recovering to a resting plateau after the previous episode of activity.

Additionally, the peak activity level and the total activity may be computed from the activity sensor signal for the entire duration of the detected non-resting episode at block 318. The duration of the non-resting activity episode, may also be computed and recorded at block 318 along with other physiological parameters measured by the IMD such as atrial and ventricular rates and heart rate turbulence.

When the deoxygenation time interval and/or recovery time interval are determined for a known, prescribed level of activity in response to user input, the time interval(s) may be used directly for computing an exercise tolerance metric at block 320. In other embodiments, time intervals may be normalized by an activity level, total activity, activity episode duration or any combination thereof. In still other embodiments, the exercise tolerance metric may be determined at block 320 by mapping deoxygenation and/or reoxygenation times as a function of activity measurements (i.e. peak activity level, total activity, activity duration) at block 320 and determining a feature of the mapped data. As such, a peak activity level, total activity, activity duration, and/or other measurements of the patient activity during the non-resting activity episode may be determined at block 318. The peak activity level may be a maximum activity measurement or a maximum level of activity sustained for a minimum interval of time.

Instead of determining a peak activity, another representative measurement of the non-resting episode may be determined. For example an overall average activity occurring between the onset of detecting non-resting activity and the end of the non-resting activity. The end of the non-resting activity may be detected as the end of a non-resting plateau of the tissue oxygenation measurement, i.e. the point at which tissue oxygenation begins to rise, or detected based on the activity sensor signal.

At block 320, the time intervals computed at blocks 310 and/or 318 may be normalized by the peak activity or other activity episode measurement determined at block 312 and 318 or a combination thereof. Alternatively, the exercise tolerance metric may be computed over a range of non-resting activity levels and trends in the exercise tolerance metric as a function of activity level can be identified by plotting the exercise tolerance metric versus activity level and comparing the plotted data at different time points.

At block 322, an exercise tolerance metric may be computed using the normalized time interval(s) or mapped data. In this way, time intervals determined for varying levels of non-resting activity can be determined and used to compute a metric of exercise tolerance that can be compared to previously determined metrics even if the peak exercise or peak sustained exercise varied between measurements. It is recognized that other methods may include different activity-level corrections of a tissue oxygenation measurement or an exercise tolerance metric computed from a tissue oxygenation measurement.

The stored exercise tolerance metric is compared to previously stored metrics to determine a trend in exercise tolerance at block 322. If the trend in exercise tolerance metrics indicates a worsening trend as determined at block 324, a notification may be generated at block 326 to notify a clinician of the worsening condition. This enables a clinician to modify current heart failure therapies. In alternative embodiments, it is recognized that a therapy delivered by the IMD may be adjusted or modified in response to detecting a worsening trend in the exercise tolerance metric.

A worsening trend will correspond generally to a decrease in a deoxygenation time interval and/or an increasing recovery time interval for a given level of exercise. In other words, the skeletal muscle tissue oxygen is quickly depleted during exercise and takes longer to recover after exercise when the patient's exercise tolerance is worsening due to a worsening heart failure condition.

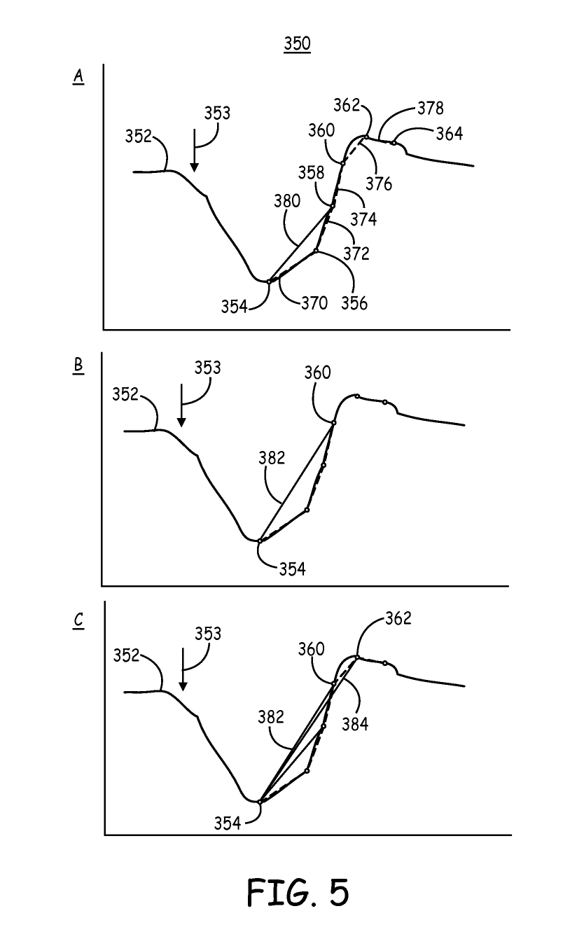

FIG. 5 is a plot 350 of a tissue oxygenation measurement 352 illustrating an alternative method for measuring a recovery curve slope for use in computing an exercise tolerance metric. After detecting a non-resting level of activity at 353, a minimum peak 354 of the tissue oxygenation measurement 352 is detected. Subsequent tissue oxygenation measurements are indicated at points 356, 358, 360, 362 and 364, which may be sampled at any desired intervals. Slopes 370, 372, 374, 376, and 378 are measured between consecutive measurements 354 through 364. Additionally, slopes 380 (panel A), 382 (panel B), and 384 (panel C) are measured between the minimum peak 354 and each subsequent sample point 358, 360, 362, respectively. Upon detecting a decrease in the consecutive slope measurements 370 through 378, a maximum slope measured between minimum peak 354 and any of the measurements 356 through 364 may be identified as a recovery slope curve.

In the example shown, a decreasing trend in the consecutive slope measurements is identified at slope 376. Slope 384 between minimum peak 354 and the measurement sample point 362, associated with the first decrease in consecutive slope measurement, is the last recovery slope measured. Slope 382 is identified as the maximum recovery slope and may be stored as a recovery curve slope for use in computing an exercise tolerance metric.

In an alternative embodiment, the first time derivative of the tissue oxygenation measurement curve 352 may be computed to allow an interval corresponding to the recovery curve (between a minimum peak 354 and a maximum peak 362 or plateau portion) to be identified. The peak of the first time derivative during the identified interval provides a measurement correlated to the rate of tissue oxygenation recovery for use in computing an exercise tolerance metric. A similar measurement technique could be applied to the deoxygenation curve (between an onset of non-resting activity 353 and minimum peak 354) for obtaining a measurement correlated to the rate of tissue deoxygenation.

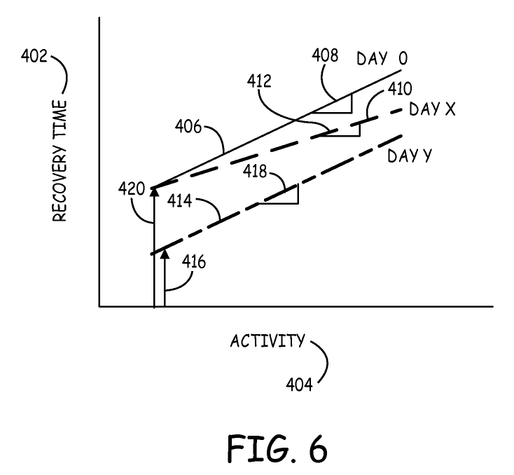

FIG. 6 is a hypothetical exercise tolerance map of an exercise tolerance metric used for monitoring a patient's heart failure condition. In this example, a recovery time interval 402 is determined for various spontaneous activity levels detected in a patient. The recovery time interval 402 is plotted as a function of activity 404 at different time points, Day 0, Day X and Day Y. Initially at Day 0, the recovery time-activity curve 406 has a slope 408 and y-offset 420. While an offset is shown measured at the lowest activity level of the recovery time curve, an offset may be measured or interpolated at a different activity level, for example a predetermined level mid-way between the highest and lowest activity levels.

An exercise tolerance metric may be determined using either or both of the slope or a y-offset of the recovery time-activity curve. At Day X, the recovery time-activity curve 410 has the same y-offset by a lower slope 412. The reduced slope of the recovery-time activity curve indicates the recovery time is shorter for the same level of activity compared to Day 0. The reduced slope of the recovery time-activity curve 410 therefore represents an improvement in the heart failure condition.

At Day Y, the recovery time-activity curve 414 has the same slope 418 as the Day 0 slope 408. However, the y-offset 416 of Day Y curve 414 is reduced compared to the y-offset 410 of Day 0. This indicates the recovery time at all activity levels is shortened at Day Y as compared to Day 0. The reduced y-offset of the recovery time-activity curve signifies an improved exercise tolerance and improving heart failure condition.

An exercise tolerance metric may therefore be computed as an activity-corrected time interval or slope computed during exercise or during post-exercise recovery. In other embodiments an exercise tolerance metric is determined as a feature of a time interval-activity level curve. Computed exercise tolerance metrics and supporting time interval, activity level, and tissue oxygenation data may be stored in the IMD and transmitted to an external device or remote patient management database for further analysis and/or display for review by a clinician.

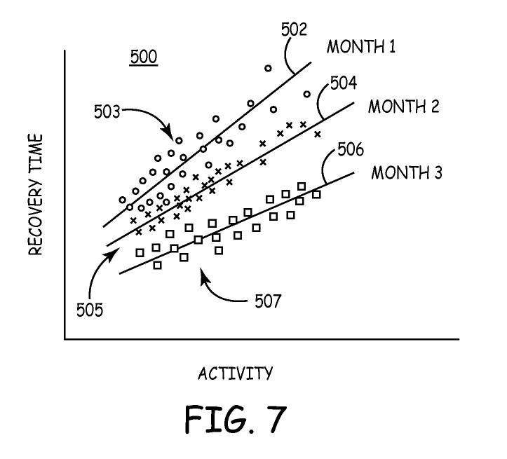

FIG. 7 is a hypothetical plot 500 of recovery time interval data acquired over three different time periods for spontaneously detected activity levels. Activity and tissue oxygenation data may be stored by the IMD or an external device or database to allow plots of determined time intervals and activity to be generated for user specified time points or intervals. For example, a clinician may request multiple, superimposed plots of time interval-activity data spanning a user-specified time period, such as one day, one week, one month, etc. In this way, multiple plots of data acquired over one day, one week, one month or other specified time period may be viewed simultaneously to observe trends in the patients exercise tolerance.

In the example of FIG. 7, recovery time interval-activity level curves 502, 504, and 506 may be generated using a best fit curve-fitting algorithm applied to recovery time interval data points 503 (circles), 505 (x's) and 507 (squares), each respectively acquired over different one month periods for spontaneously detected patient activity levels. A clinician may observe trends in the patient's exercise tolerance by viewing data plotted over different intervals of time and superimposed on a common plot 500. Such observation may indicate if the patient is responding well to a therapy and may be used for making therapy adjustments.

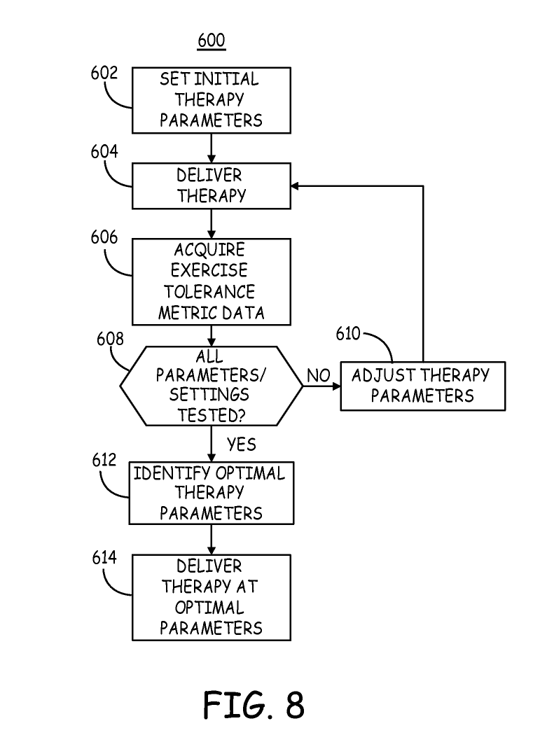

FIG. 8 is a flow chart of a method 600 for optimizing a heart failure therapy. At block 602, one or more therapy parameters are set to initial values. The heart failure therapy being optimized may be a drug therapy, taken orally or delivered automatically by an IMD, and the therapy parameter being optimized is a dosage level. Alternatively, the heart failure therapy may be cardiac resynchronization therapy (CRT) delivered by the IMD. Therapy parameters being adjusted may include an atrial-ventricular (AV) timing interval and/or a ventricular-ventricular (VV) timing interval. Other heart failure therapies may be delivered by an implanted device, an external device, by a clinician, or self-administered and the therapy control parameters being adjusted and optimized will depend on the particular therapy.

At block 604, the therapy being optimized is delivered at the initial therapy control parameter setting(s). Data for computing an exercise tolerance metric is acquired for a specified monitoring time interval, e.g. one hour, one day, one week, one month, etc, at block 606. As described above, acquired data will include at least patient activity data and time interval data associated with deoxygenation during non-resting activity and/or oxygen recovery subsequent to non-resting activity. The activity may be spontaneous patient activity or prescribed activity performed, which may be performed under controlled, supervised conditions.

After acquiring data for the specified monitoring interval, the next parameter setting to be tested is set at block 610 until all desired test parameters and associated test settings have been applied as determined at block 608. At block 612, the exercise tolerance metric data is analyzed to identify the therapy delivery parameter setting(s) that resulted in the greatest tolerance to exercise, e.g. as evidenced by improved deoxygenation and/or recovery times for a given level of patient activity. A multi-variate analysis may be performed to identify a combination of therapy parameter settings that results in a peak exercise tolerance metric for a given patient.

The therapy is delivered at the optimal setting(s) at block 614. Method 600 may return to block 612 to continue acquiring exercise tolerance data in a closed-loop feedback manner for device-delivered therapies. Adjustments to therapy delivery control parameters may be made as needed in response to a decline in exercise tolerance or to maintain optimal exercise tolerance.

Thus, an implantable medical device and associate method for monitoring a heart failure patient have been presented in the foregoing description with reference to specific embodiments. It is appreciated that various modifications to the referenced embodiments may be made without departing from the scope of the disclosure as set forth in the following claims.

1. A method for monitoring a heart failure patient, the method comprising:
sensing a first signal responsive to oxygen availability in an extravascular volume of skeletal muscle tissue in the patient and computing a tissue oxygenation measurement using the first signal;
detecting a change in the tissue oxygenation measurement;
computing a time interval corresponding to the detected change in muscle tissue oxygenation; and
detecting if a heart failure condition is worsening in response to the computed time interval.

2. The method of claim 1 further comprising:
sensing a second signal responsive to physical activity of a patient;
detecting an episode of non-resting patient activity in response to the first signal; and
detecting the change in the tissue oxygenation measurement subsequent to detecting the episode of non-resting patient activity.

3. The method of claim 2, wherein detecting the change in the tissue oxygenation measurement comprises detecting a first level of tissue oxygenation and detecting a second level of tissue oxygenation during the episode, the second level being lower than the first level.

4. The method of claim 3, further comprising detecting a third level of tissue oxygenation corresponding to a plateau during the episode, the second level being intermediate to the first level and the third level,
the detected change being the change between the first level and the third level.

5. The method of claim 2, wherein detecting the change in the tissue oxygenation measurement comprises detecting a first level of tissue oxygenation corresponding to a minima reached during the detected non-resting activity; and
detecting a second level of tissue oxygenation greater than the first level and occurring after the first level of tissue oxygenation.

6. The method of claim 5, further comprising detecting a third level of tissue oxygenation corresponding to a plateau higher than the first level and occurring after the first level, the second level being intermediate to the first level and the third level,
the detected change being the change from the first level to one of the second and the third level.

7. The method of claim 2, further comprising:
determining a feature of the first signal as a first measure of activity level for the episode;
detecting a next episode of non-resting activity having a next measure of activity level equal to the first measure of activity level; and
computing a next time interval corresponding to a change in tissue oxygenation in response to detecting the next episode;
wherein detecting if the heart failure condition is worsening comprises determining a trend using the time interval and the next time interval.

8. The method of claim 7 wherein the feature being one of a maximum peak activity level, a maximum sustained activity level, and an average activity level.

9. The method of claim 2, further comprising:
determining a feature of the first signal as a first measure of activity level for the episode;
detecting a next episode of non-resting activity having a next measure of activity level different than the first measure;
computing a next time interval corresponding to a change in tissue oxygenation in response to detecting the next episode;
plotting the computed time interval and the next time interval as a function of the respective first measure and next measure;
wherein detecting if the heart failure condition is worsening comprises determining a trend in the plotted time interval and plotted next time interval.

10. The method of claim 2 further comprising:
plotting the time interval and a previously determined time interval as a function of activity;
determining a best fit curve of the plotted time interval and previously determined time interval;
wherein detecting if the heart failure condition is worsening comprises comparing a feature of the curve to a feature of a previously determined best fit curve.

11. The method of claim 1 further comprising;
delivering a heart failure therapy;
adjusting a parameter controlling delivery of the heart failure therapy from a first setting to a second setting;
computing the time interval when the heart failure therapy is delivered using the first setting and when the heart failure therapy is delivered using the second setting;
identifying an optimal setting of the parameter using the computed time intervals; and
delivering the therapy using the optimal setting.

12. The method of claim 1 wherein detecting the change in the tissue oxygenation measurement comprises detecting a tissue oxygenation minima and measuring at least one subsequent tissue oxygenation measurement greater than the minima;
wherein computing the time interval comprises computing a recovery curve slope using the at least one subsequent tissue oxygenation measurement.

13. An implantable medical device for monitoring a heart failure patient, the device comprising:
a tissue oxygenation sensor sensing a first signal responsive to oxygen availability in an extravascular volume of skeletal muscle tissue in the patient;
a processor receiving the first signal and configured to compute a tissue oxygenation measurement using the first signal, detect a change in the tissue oxygenation measurement compute a time interval corresponding to the detected change in muscle tissue oxygenation; and detect if a heart failure condition is worsening in response to the computed time interval.

14. The device of claim 13 further comprising an activity sensor generating a second signal responsive to physical activity of a patient;
the processor receiving the second signal and further configured to detect an episode of non-resting patient activity in response to the second signal and detect the change in the tissue oxygenation measurement subsequent to detecting the episode of non-resting patient activity.

15. The device of claim 14, wherein detecting the change in tissue oxygenation measurement comprises detecting a first level of tissue oxygenation and detecting a second level of tissue oxygenation during the episode, the second level being lower than the first level.

16. The device of claim 15, wherein the processor is further configured to detect a third level of tissue oxygenation corresponding to a plateau during the episode, the second level being intermediate to the first level and the third level,
the detected change being the change between the first level and the third level.

17. The device of claim 14, wherein detecting the change in the tissue oxygenation measurement comprises detecting a first level of tissue oxygenation corresponding to a minima reached during the detected non-resting activity; and
detecting a second level of tissue oxygenation greater than the first level and occurring after the first level of tissue oxygenation.

18. The device of claim 17, wherein the processor is further configured to detect a third level of tissue oxygenation corresponding to a plateau higher than the first level and occurring after the first level, the second level being intermediate to the first level and the third level,
the detected change being the change from the first level to the second level.

19. The device of claim 14, wherein the processor is further configured to:
determine a feature of the second signal as a first measure of activity level for the episode;
detect a next episode of non-resting activity having a next measure of activity level equal to the first measure of activity level; and
compute a next time interval corresponding to a change in tissue oxygenation in response to detecting the next episode;
wherein detecting if the heart failure condition is worsening comprises determining a trend of the time interval and the next time interval.

20. The device of claim 19 wherein the feature being one of a maximum peak activity level, a maximum sustained activity level, and an average activity level.

21. The device of claim 14 wherein the processor is further configured to:
determine a feature of the first signal as a first measure of activity level for the episode;
detect a next episode of non-resting activity having a next measure of activity level different than the first measure;
compute a next time interval corresponding to a change in tissue oxygenation in response to detecting the next episode;
plotting the computed time interval and the next time interval as a function of the respective first measure and next measure;
wherein detecting if the heart failure condition is worsening comprises determining a trend in the plotted time interval and plotted next time interval.

22. The device of claim 14 wherein the processor is further configured to:
plot the time interval and a previously determined time interval as a function of activity;
determine a best fit curve of the plotted time interval and previously determined time interval;
wherein detecting if the heart failure condition is worsening comprises comparing a feature of the curve to a feature of a previously determined best fit curve.

23. The device of claim 13 further comprising;
a therapy delivery module;
a control module for adjusting a therapy control parameter used by the therapy delivery module from a first setting to a second setting;
wherein the processor is configured to compute the time interval when the heart failure therapy is delivered using the first setting and when the heart failure therapy is delivered using the second setting; identify an optimal setting of the parameter using the computed time intervals;
wherein the control module uses the optimal setting for controlling the therapy delivery module to deliver the therapy.

24. The device of claim 13 wherein the processor is configured to detect the change in the tissue oxygenation measurement by detecting a tissue oxygenation minima and measuring at least one subsequent tissue oxygenation measurement greater than the minima;
wherein computing the time interval comprises computing a recovery curve slope using the at least one subsequent tissue oxygenation measurement.
